SEQUENCE LISTING (12 sequences)

SEQ ID NO: 1 - DNA, 42 bp, Artificial
  Synthetic oligonucleotide; partial ss sequence of pssXB (AG30) vector; partially complementary to SEQ ID NO2
gggccgcaggctccccctcccccaccaccccccccttcctgc

SEQ ID NO: 2 - DNA, 41 bp, Artificial sequence
  Synthetic oligonucleotide ; partial ss sequence of pssXB (AG30) vector; partially complementary to SEQ ID NO1
ggccgcaggaaggggggggtggtgggggagggggagcctgc

SEQ ID NO: 3 - DNA, 15 bp, Artificial sequence
  Synthetic oligonucleotide PCR primer to AG34 (or) rev34
caggctccccctccc

SEQ ID NO: 4 - DNA, 15 bp, Artificial sequence
  Synthetic oligonucleotide PCR primer to AG34 (or) rev34
caggaaggggggggt

SEQ ID NO: 5 - DNA, 34 bp, Artificial sequence
  Synthetic oligonucleotide directed to E.coli supF gene
aggaaggggggggtggtgggggagggggagcctg

SEQ ID NO: 6 - DNA, 85 bp, Artificial sequence
  Oligonucleotide forming a single stranded AG30, MboII cleavage site, and double stranded stem (stem-loop structure)
ggtcggcggccttgaacagcggccgcaggaaggggggggtggtgggggagggggagcctgcggccgctcttcaaggccgccgacc

SEQ ID NO: 7 - DNA, 88 bp, Artificial sequence
  Partial sequence of pssXB (AG30) vector; complementary to SEQ ID NO8
ctaggtcggcggccttgaagagcggccgcaggctccccctcccccaccaccccccccttcctgcggccgctcttcaaggccgccgacc

SEQ ID NO: 8 - DNA, 88 bp, Artificial sequence
  partial sequence of pssXB (AG30) vector; complementary to SEQ ID NO7
ggtcggcggccttgaagagcggccgcaggaaggggggggtggtgggggagggggagcctgcggccgctcttcaaggccgccgacctag

SEQ ID NO: 9 - DNA, 30 bp, Artificial sequence
  Synthetic oligonucleotide directed to E.coli supF gene
aggaaggggggggtggtgggggagggggag

SEQ ID NO: 10 - DNA, 30 bp, Artificial sequence
  G rich polypurine sequence targeted by AG30; complementary to SEQ ID NO11
gagggggagggggtggtggggggggaagga

SEQ ID NO: 11 - DNA, 30 bp, Artificial sequence
  complementary strand to SEQ ID NO10
tccttccccccccaccaccccctccccctc

SEQ ID NO: 12 - DNA, 34 bp, Artificial sequence
  Synthetic oligonucleotide; AG30 sequence in reverse orientation
aggctccccctcccccaccaccccccccttcctg

(19) United States
(12) Patent Application Publication
(10) Pub. No.: US 2003/0148352 A1
(43) Pub. Date: Aug. 7, 2003

(54) Intracellular generation of single-stranded DNA

(75) Inventor: Peter M. Glazer, Guilford, CT (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Yale University

(21) Appl. No.: 10/319,369

(22) Filed: Dec. 13, 2002

Related U.S. Application Data
(60) Provisional application No. 60/340,803, filed on Dec. 14, 2001.

(51) Int. Cl. C12Q 1/68; C12N 15/00; C12N 15/85

(52) U.S. Cl. 435/6; 435/455; 435/320.1

(57) ABSTRACT

Methods for intracellularly generating single stranded DNA molecules that are active in mediating triplex-dependent or independent chromosomal events are provided herein. The method is based on the discovery that one can introduce viral vectors or plasmids into the cells, where they generate within the cells to be engineered the single stranded DNA molecules that bind to the target chromosomal DNA to form a triplex, which may induce a desired mutation, and/or be recombinagenic and induce a change to the chromosomal DNA by incorporation of a donor DNA molecule. The vectors or plasmid not only encode the TFO and optionally the donor DNA, but also a reverse transcriptase, and optionally, a restriction enzyme, which is present in the preferred embodiment as a fusion protein which reverse transcribes the RNA encoded by the vector or plasmid, then cleaves it at a restriction enzyme site to yield a single stranded DNA. The single stranded DNA may be produced directly, or initially as a double stranded stem-single stranded loop structure, which is then cleaved to yield the single stranded DNA.

### CROSS-REFERENCE TO RELATED APPLICATIONS

This application claims the benefit of U.S. Provisional Application No. 60/340,803, filed Dec. 14, 2001.

The United States Government has certain rights in this invention by virtue of National Institutes of Health Grant Nos. RO1 GM54731 and RO1 CA64186.

### BACKGROUND OF THE INVENTION

Gene therapy can be defined by the methods used to introduce heterologous DNA into a host cell or by the methods used to alter the expression of endogenous genes within a cell. As such, gene therapy methods can be used to alter the phenotype and/or genotype of a cell. One example is the field of antisense therapy. In antisense therapy, a nucleic acid molecule is introduced into a cell, where it hybridizes or binds to the mRNA encoding a specific protein. The binding of the antisense molecule to an mRNA species decreases the efficiency and rate of translation of the mRNA.

Methods which alter the genotype of a cell typically rely on the introduction into the cell of an entire replacement copy of a defective gene, a heterologous gene, or a small nucleic acid molecule such as an oligonucleotide, to treat human, animal and plant genetic disorders. The introduced gene or nucleic acid molecule, via random integration, supplements the endogenous gene. These approaches require complex delivery systems to introduce the replacement gene into the cell, such as genetically engineered viruses, or viral vectors. Gene therapy is being used on an experimental basis to treat well known genetic disorders in humans such as retinoblastoma, cystic fibrosis, and sickle cell anemia. However, in vivo efficiency is low due to the limited number of recombination events actually resulting in integration of the defective gene. Moreover, genes can integrate into portions of the chromosome where expression is limited or can lead to deleterious effects, such as induction of an oncogene.

Targeted modification of the genome by gene replacement is of value as a research tool and in gene therapy. However, while facile methods exist to introduce new genes into mammalian cells, the frequency of homologous integration is limited (Hanson et al., (1995) Mol. Cell. Biol. 15(1), 45-51), and isolation of cells with site-specific gene insertion typically requires a selection procedure (Capecchi, M. R., (1989) Science 244(4910), 1288-1292). Site-specific DNA damage in the form of double-strand breaks produced by rare cutting endonucleases can promote homologous recombination at chromosomal loci in several cell systems, but this approach requires the prior insertion of the recognition sequence into the locus.

Since the initial observation of triple-stranded DNA many years ago by Felsenfeld et al., J. Am. Chem. Soc. 79:2023 (1957), oligonucleotide-directed triple helix formation has emerged as a valuable tool in molecular biology. Current knowledge suggests that oligonucleotides can bind as third strands of DNA in a sequence specific manner in the major groove in polypurine/polypyrimidine stretches in duplex DNA. In one motif, a polypyrimidine oligonucleotide binds in a direction parallel to the purine strand in the duplex, as described by Moser and Dervan, Science 238:645 (1987), Praseuth et al., Proc. Natl. Acad. Sci. USA 85:1349 (1988), and Mergny et al., Biochemistry 30:9791 (1991). In the alternate purine motif, a polypurine strand binds anti-parallel to the purine strand, as described by Beal and Dervan, Science 251:1360 (1991). The specificity of triplex formation arises from base triplets (AAT and GGC in the purine motif) formed by hydrogen bonding; mismatches destabilize the triple helix, as described by Mergny et al., Biochemistry 30:9791 (1991) and Beal and Dervan, Nuc. Acids Res. 11:2773 (1992).

Single stranded DNA (ssDNA) is useful for several molecular biology techniques. Single stranded DNA can bind to double stranded DNA in a sequence-specific manner to form triple helices (Giovannangeli et al., 2000, Curr. Opin. Mol. Ther., 2, 288-296; Chan et al., 1997, J. Mol. Med. 75, 267- 282). Such ssDNA is hereinafter referred to as a triplex forming oligonucleotide, or TFO. Triple helix formation has been shown to suppress gene expression (Faria et al., 2000, Proc. Natl. Acad. Sci. USA, 97, 3862-3867; Kim et al., 1998, Biochemistry, 37, 2299-304) and has been shown to mediate targeted genome modification in mammalian cells via directed mutagenesis or induced recombination (Luo et al., 2000, Proc. Natl. Acad. Sci. USA 97, 9003-9008; Vasquez et al., 2000, Science, 290, 530-533). The ability of TFOs to stimulate recombination has been shown to depend on XPA and Rad51 (Datta et al., 2001, J. Biol. Chem., 27, 18018-18023; Faruqi et al., 2000, Mol. Cell. Biol., 20, 990-1000), factors involved in nucleotide excision repair and homologous recombination, respectively. These results are consistent with studies demonstrating that triplex structures provoke DNA repair (Wang et al., 1996, Science, 271, 802-805).

U.S. Pat. Nos. 5,962,426 and 6,303,376 to Glazer et al., describe the use of TFOs to induce site specific mutations and/or recombination of donor oligonucleotides, for use in gene therapy. Studies demonstrate that a G-rich 30-mer TFO (AG30), when transfected into a mouse fibroblast cell line (FL-10), could induce recombination between direct repeat copies of the herpes simplex virus thymidine kinase (TK) gene in a chromosomal substrate in which the target site for triplex formation was situated between the genes (Luo et al., 2000, Proc. Natl. Acad. Sci., USA, 97, 9003-9008). The AG30 TFO was 3′-end protected from degradation by modification with a propylamine group and transfected into cells either by co-mixture with cationic lipids or via direct microinjection. Although lipid-mediated transfection yielded specific and detectable induction of recombination, microinjection yielded a 300-fold higher frequency of recombinants. Additionally, high affinity, triplex-forming oligonucleotides and methods for use have been described and used to form a triple-stranded nucleic acid molecule with a specific DNA segment of a target DNA molecule. Upon formation of the triplex, the binding of the oligonucleotide stimulated mutagenesis within or adjacent to the target sequence using cellular DNA synthesis or repair mechanisms in vivo, without recombination.

The disadvantage of these methods is that they require introduction of the TFOs and, optionally, donor DNA for recombination, into the cells to be engineered. Most methods for delivery in vivo have a low degree of efficiency.

It is therefore an object of the present invention to provide a means for delivery of providing large quantities of TFOs and/or donor DNA for recombination, within cells.

### SUMMARY OF THE INVENTION

Methods for intracellularly generating single stranded DNA molecules that are active in mediating triplex-dependent or independent chromosomal events are provided herein. The method is based on the discovery that one can introduce viral vectors or plasmids into the cells, where they generate within the cells to be engineered the single stranded DNA molecules that bind to the target chromosomal DNA to form a triplex, which may induce a desired mutation, and/or be recombinagenic and induce a change to the chromosomal DNA by incorporation of a donor DNA molecule. The vectors or plasmid not only encode the TFO and optionally the donor DNA, but also a reverse transcriptase, and optionally, a restriction enzyme, which is present in the preferred embodiment as a fusion protein which reverse transcribes the RNA encoded by the vector or plasmid, then cleaves it at a restriction enzyme site to yield a single stranded DNA. The single stranded DNA may be produced directly, or initially as a double stranded stem-single stranded loop structure, which is then cleaved to yield the single stranded DNA.

Once produced in vivo, these high affinity, triplex-forming oligonucleotides are targeted to chromosomal sequences where they bind to and induce a chromosomal event. Such events include recombination, gene conversion, nucleotide substitution, nucleotide deletion, nucleotide insertion, changing or correcting a genetic defect at a chromosomal site. The mutation or recombination results in a change which activates, inactivates, or alters the activity and function of the target gene. If the target gene contains a mutation that is the cause of a genetic disorder, then the oligonucleotide is useful for mutagenic or recombinational repair that restores the DNA sequence of the target gene to normal. If the target gene is a viral gene needed for viral survival or reproduction or an oncogene causing unregulated proliferation, such as in a cancer cell, then the mutagenic oligonucleotide is useful for causing a mutation that inactivates the gene to incapacitate or prevent reproduction of the virus or to terminate or reduce the uncontrolled proliferation of the cancer cell. The mutagenic oligonucleotide is also a useful anti-cancer agent for activating a repressor gene that has lost its ability to repress proliferation.

The triplex-forming oligonucleotide can also be used as a molecular biology research tool to cause targeted mutagenesis in a cell. Targeted mutagenesis is useful for targeting a normal gene and for the study of mechanisms such as DNA repair or any type of genomic functionality. Targeted mutagenesis of a specific gene in an animal oocyte, such as a mouse oocyte, provides a useful and powerful tool for genetic engineering for research and therapy and for generation of new strains of “transmutated” animals and plants for research and agriculture. Triplex-forming oligonucleotides are particularly useful as molecular research tools in the field of functional genomics.

### BRIEF DESCRIPTION OF THE DRAWINGS

FIG. 1. Design of the vector system to produce ssDNA in mammalian cells. (A) The vector pssXA expresses an RT-MboII fusion protein driven by a Rous sarcoma virus (RSV) promoter. It also carries a neomycin-resistance gene as a selectable marker. pssXB (AG30) is engineered to express from a cytomegalovirus (CMV) promoter a transcript containing a desired sequence insert situated within NotI sites. The transcript is designed to also incorporate a core promoter site for MoMuLV RT, thereby allowing generation of a cDNA containing one strand of the insert sequence. (B) Partial DNA sequence of the pssXB(AG30) vector. The sequence of the fragment inserted into the Not I sites of pssXB to produce pssXB(AG30) is shown in upper case letters, with the AG30 TFO sequence in bold. The MboII and NotI sites are indicated by underlining and italics, respectively. (C) Stem-loop structure predicted to be formed by the cDNA produced upon reverse transcription of the pssXB(AG30)-derived transcript. The MboII recognition site predicted to form in the stem and the expected sites of MboII cleavage are indicated. The reverse transcriptase can be fused to any restriction enzyme known in the art. For example, the enzyme may be selected from the non-limiting group: Eco RI, Mbo I, Hind III, Bam HI, Nde I, Bgl I, Not I, Pst I, Sac I, Sce I, Ssp I, and Xba I. The selection of enzyme will coincide with the site to be cleaved. It may be desirable to fuse the reverse transcriptase to a rare cutting enzyme. For example an 18 base pair cutter, such as Sce I, to limit digestion of certain chromosomal DNA. (D) Predicted 34 nucleotide ssDNA product (AG34) to be produced from the combined transfection of pssXA and pssXB (AG30), based on the proposed pattern of MboII cleavage in (C). The 30 nucleotide (nt) AG30 TFO sequence contained within AG34 is underlined, distinguishing this portion of AG34 from the extra 4 nt at the 3′ end.

FIG. 2. (A) Sequences of the ssDNA products. AG34 and rev34, expected to be produced by pssXA plus pssXB (AG30) and by pssXA plus pssXB(rev), respectively, in comparison with AG30 (indicated by the underlining). (B) Target substrate designed to investigate induction of intrachromosomal recombination by TFOs. LTK⁻ cells carrying, at a single chromosomal locus, two mutant copies of the TK gene as direct repeats flanking a polypurine third-strand binding site were used to test the ability of vector-generated ssDNAs to mediate triplex formation and recombination induction. The TK genes carry inactivating mutations consisting of XhoI linker insertions at the indicated positions. Potential recombinants are identified as TK+ clones growing selective HAT-containing medium.

### DETAILED DESCRIPTION OF THE INVENTION

The method described herein provides single stranded DNA molecules that are generated intracellularly and are active in mediating triplex-dependent and/or recombinagenic chromosomal events within the cells and/or the cellular compartments to be treated. There are three basic situations:

(1) where the single stranded DNA binds to the target chromosomal DNA to form a triplex which is sufficient to induce a site specific mutation;

(2) wherein the single stranded DNA binds to the target chromosomal DNA and induces recombination with the chromosomal DNA; and

(3) a combination of (1) and (2), wherein the single stranded DNA both forms a triplex and recombines with the target chromosomal DNA. In this case, the single stranded DNA may consist of the triplex forming sequence alone, the triple helix forming sequence linked to the recombinagenic sequence, or there can be two single stranded DNAs, one forming a triplex and the other which is recombinagenic.

These oligonucleotides are produced within the cells to be engineered by providing a vector or plasmid which generates not only the oligonucleotides in the cells, but also a fusion protein which is both a reverse transcriptase and a restriction enzyme.

Although the prior art teaches that one can use triplex forming oligonucleotides alone or in combination with recombinagenic oligonucleotides, studies had to be performed to determine if these could be introduced into cells as viral vectors or plasmids, along with the means for processing of the oligonucleotides, before one would know that the single stranded DNA would be taken up in the nucleus and be effective to introduce changes into the chromosomal DNA.

The methods described herein are highly specific and efficient, and result in much higher rates of genetic engineering, than exogenous delivery of oligonucleotides to the cells.

I. ssDNA Molecules Forming Triple Helices or Recombining into a Target Chromosomal Sequence.

As noted above, single stranded DNA molecules can be either triplex forming oligonucleotides, recombinagenic oligonucleotides, or a combination thereof.

A. Triple Helix Forming Oligonucleotides TFOs are defined as triplex-forming oligonucleotides which bind as third strands to duplex DNA in a sequence specific manner. It is preferred that the “TFOs” be single stranded DNA. Single stranded DNA may, or may not, form third strands with duplex DNA. Single stranded DNA is noted below as 1) being able to form triple helices with duplex DNA, 2) be recombined into a target chromosomal sequence, or 3) serve as a template for in vivo repair of a chromosomal segment. Such events are further described below.

The preferred conditions under which a triple-stranded structure will form are standard assay conditions for in vitro mutagenesis and physiological conditions for in vivo mutagenesis. (See for example, Moser and Dervan, Science 238:645 (1987); Praseuth et al., Proc. Natl. Acad. Sci. USA 85:1349 (1988); Mergny et al., Biochemistry 30:9791 (1991); Beal and Dervan, Science 251:1360 (1991); Mergny et al., Biochemistry 30:9791 (1991) and Beal and Dervan, Nuc. Acids Res. 11:2773 (1992), which are incorporated by reference herein.)

A useful measure of triple helix formation is the equilibrium dissociation constant, K_{d}, of, the triplex, which can be estimated as the concentration of oligonucleotide at which triplex formation is half-maximal. Preferably, the oligonucleotide has a binding affinity for the target sequence in the range of physiologic interactions. The preferred oligonucleotide has a K_{d} less than or equal to approximately 10⁻⁷ M. Most preferably, the K_{d} is less than or equal to 2×10⁻⁸ M in order to achieve significant intracellular interactions. A variety of methods are available to determine the K_{d} of an oligonucleotide/target pair.

In one embodiment, a high affinity oligonucleotide (K_{d}≦2×10⁻⁸) which forms a triple strand with a specific DNA segment of a target gene DNA is generated. It is preferable that the Kd for the high affinity oligonucleotide is less than or equal to 2×10⁻⁶. It is more preferable that the K_{d} for the high affinity oligonucleotide is less than or equal to 2×10⁻⁷. It is still more preferable that the K_{d} for the high affinity oligonucleotide be below 2×10⁻⁸. It is most preferable that the K_{d} for the high affinity oligonucleotide be below 2×10⁻⁹.

The oligonucleotide binds to a target sequence within a target gene or target region of a chromosome, forming a triplex region. Preferably, the target region of the double-stranded molecule contains or is adjacent to a defective or essential portion of a target gene, such as the site of a mutation causing a genetic defect, a site causing oncogene activation, or a site causing the inhibition or inactivation of an oncogene suppressor. Most preferably, the gene is a human gene.

Preferably, the oligonucleotide is a single-stranded nucleic acid molecule between 7 and 40 nucleotides in length, most preferably 10 to 30 nucleotides in length for in vivo chromosomal modifications. The base composition is preferably homopurine or homopyrimidine. Alternatively, the base composition is polypurine or polypyrimidine. However, other compositions are also useful.

The nucleotide sequence of the oligonucleotides herein described is selected based on the sequence of the target sequence, the physical constraints imposed by the need to achieve binding of the oligonucleotide within the major groove of the target region, and the need to have a low dissociation constant (K_{d}) for the oligonucleotide/target sequence. The oligonucleotides will have a base composition which is conducive to triple-helix formation and will be generated based on one of the known structural motifs for third strand binding. In the motif used in the Example which follows (the anti-parallel purine motif), a G is used when there is a GC pair and an A is used when there is a AT pair in the target sequence. When there is an inversion, a CG or TA pair, another residue is used, for example, a T is used for a TA pair. A review of base compositions for third strand binding oligonucleotides is provided in U.S. Pat. No. 5,422,251.

Preferably, the oligonucleotide binds to the target nucleic acid molecule under conditions of high stringency and specificity. Most preferably, the oligonucleotides bind in a sequence-specific manner within the major groove of duplex DNA. Reaction conditions for in vitro triple helix formation of an oligonucleotide probe or primer to a nucleic acid sequence vary from oligonucleotide to oligonucleotide, depending on factors such as oligonucleotide length, the number of G:C and A:T base pairs, and the composition of the buffer utilized in the binding reaction. An oligonucleotide substantially complementary, based on the third strand binding code, to the target region of the double-stranded nucleic acid molecule is preferred. As used herein, an oligonucleotide is said to be substantially complementary to a target region when the oligonucleotide has a base composition which allows for the formation of a triple-helix with the target region. As such, an oligonucleotide is substantially complementary to a target region even when there are non-complementary bases present in the oligonucleotide. There are a variety of structural motifs available which can be used to determine the nucleotide sequence of a substantially complementary oligonucleotide.

B. Recombinagenic or Donor DNA Molecules

Recombinagenic donor DNA fragments are homologous to the target sequence. The donor molecules are preferably between 35 and 1500 nucleotides in length; more preferably between 50 and 500 nucleotides in length. It is understood in the art that the greater the number of homologous positions with the target DNA, the greater the probability that the fragment will be recombined into the target sequence, target region, or target site. The term “recombinagenic” as used herein, is used to define a DNA fragment, oligonucleotide, or composition as being able to recombine into a target site or sequence.

The recombinagenic donor DNA can be generated from the vector or plasmid as a separate DNA molecule from the triplex forming DNA molecule or linked to the triplex forming oligonucleotide via a mixed sequence linker. The nucleotide linker is variable, depending upon the location of the chromosomal change desired in relation to the triplex formation. However, it is preferred that the linker be between 1 and 100 nucleotides in length. It is still more preferable that the linker be between 1 and 15 nucleotides in length. The DNA donor and the TFO can also be directly joined (i.e. without a linker sequence).

This “donor-linked” arrangement facilitates target site recognition via triple helix formation while at the same time positioning the donor fragment for possible recombination and information transfer. This strategy is also intended to exploit the ability of a triplex, itself, to provoke DNA repair, potentially increasing the probability of recombination with the homologous donor DNA. A plasmid based system incorporating homologous fragments tethered to sequence specific TFOs, indicator bacteria, and a plasmid vector containing a mutated version of a reporter gene, was used in conjunction with human cell extracts (i.e., not in cells) to promote targeted recombination (Datta et al., 2001, J. Biol. Chem., 27, 18018-18023; Chan et al., 1999, J. Biol. Chem., 274, 17, 11541-11548). A TFO was tethered to a donor DNA fragment homologous to a region of a target gene via a mixed sequence linker. In the bi-functional A-AG30 molecule, the donor fragment, A, consisted of a single-strand of length 40 that was homologous to positions in the target gene except at position 144, where the sequence matched that of the functional gene, thereby allowing for screening/selection of the desired phenotype.

The ability of triplex formation to promote recombination with human cell-free extracts has been tested in samples in which AG30 and a donor oligonucleotide were not linked, but rather were simply co-mixed as separate molecules together with the plasmid substrate. This resulted in an increased level of recombination, at a frequency of 40×10⁻⁵, almost as high as that produced by the linked A-AG30. This result provided further evidence that a TFO can stimulate recombination between a donor fragment and target locus. In addition, because the donor fragment in this case is separate from the TFO, the result specifically demonstrates a role for the TFO in stimulating recombination that is distinct from its ability to deliver a tethered donor fragment to the target site.

II. Reverse Transcriptase-Restriction Enzyme Fusion Proteins

The vectors or plasmids which encode the single stranded DNA forming a triplex or donor DNA must also include means for transcribing the DNA from an RNA, and in some embodiments, a means for cleaving the oligonucleotides for use as triplex forming oligonucleotides or recombinagenic donor DNA. In the preferred embodiment, the vector or plasmid encodes a reverse transcriptase-restriction enzyme fusion. It will be understood of course that the reverse transcriptase and restriction enzymes can also be expressed as separate enzymes.

A. Reverse Transcriptase

Many reverse transcriptase enzymes and the nucleotide sequences encoding them are commercially available and may be used in the methods provided herein. Reverse transcriptase enzymes include, but are not limited to AMV-reverse transcriptase (Avian Myeloblastosis Virus) and M-MuLV reverse transcriptase. Viral reverse transcriptases (e.g., MuLV and AMV) may also be used. Thermostable DNA polymerases exhibiting intrinsic reverse transcriptase activity may be used.

B. Restriction Enzymes

Any restriction enzyme known in the art may be used in the methods described herein. For example, the enzyme may be selected from the non-limiting group: Eco RI, Mbo I, Hind III, Bam HI, Nde I, Bgl I, Not I, Pst I, Sac I, Sce I, Ssp I, and Xba I. Furthermore, additional commercially available type II restriction enzymes and certain intron and intein encoded endonucleases may be used. The selection of enzyme will coincide with the site to be cleaved. It may be desirable to use a rare cutting enzyme. For example an 18 base pair cutter, such as Sce I, to limit digestion of certain chromosomal DNA. DNA cleavage using RecA assisted restriction endonucleases (RARE) may also be used, thereby significantly limiting restriction digests of chromosomal DNA.

III. Vectors/Plasmids for Delivery of Oligonucleotides

By “vector”, or “plasmid”, is meant any autonomous genetic element capable of directing the synthesis of a protein, and/or mRNA transcript, encoded by the vector. Such vectors are known to those skilled in the art. By “vector” is meant a polynucleotide molecule, preferably a DNA molecule derived, for example, from a plasmid, bacteriophage, or plant virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome.

The vector optionally contains means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin.RTM.) and the hph gene which confers resistance to the antibiotic hygromycin B.

Various vector systems are well known in the art. For example, such systems include, but are not limited to, adenoviral vector systems, adeno-associated vector systems, and retroviral vector systems.

Plasmids may also be used.

IV. Methods and Compositions for Treatment

Any eukaryotic cell in which the vector is capable of generating the ssDNA can be engineered using the system described above. In the preferred embodiment, the cells are in a tissue or more preferably an animal. The most preferred animal is a human. Suitable cell lines can include, for example, CV-1 cells, COS cells, or yeast cells. Cells can also be prokaryotic in origin.

There are a number of genetic diseases or defects which can be treated using the system. For example, these may be diseases in which there is a defective gene such as thalassemia, cystic fibrosis, SCIDS, hemophilia, and sickle cell anemia. The method can also be used to inactivate genes, especially genes involved in cancer and in viral diseases, where the targeted DNA is an oncogene or viral gene.

Typically the single stranded DNA molecule that is produced in the cell, or the nuclear compartment of the cel, is recombined into a target chromosomal sequence. The induction of targeted recombination is best served, for example, to correct a mutation in a target gene that is the cause of a genetic disorder. Alternatively, if the target gene is a viral gene needed for viral survival or reproduction or an oncogene causing unregulated proliferation, such as in a cancer cell, then the recombinagenic TFOs may be useful for inducing a mutation or correcting the mutation, by homologous recombination, thereby inactivating the gene to incapacitate or prevent reproduction of the virus or to terminate or reduce the uncontrolled proliferation of the cancer cell. The binding of the oligonucleotide to the target region of a particular genetic sequence stimulates recombination in mammalian cells at a chromosomal locus. For example, it has been shown that recombination is stimulated at chromosomal loci containing two tandem copies of the herpes simplex virus thymidine kinase gene, following direct intranuclear microinjection of the oligonucleotides. (Luo, Z et al. (2000) Proc. Natl. Acad. Sci. USA 97(16), 9003-9008)).

Preferably, the donor oligonucleotides and/or the viral vectors and/or plasmids from which they are generated, are dissolved in a physiologically-acceptable carrier, such as an aqueous solution or are incorporated within liposomes, and the carrier or liposomes are injected into the organism undergoing genetic manipulation, such as an animal requiring gene therapy or anti-viral therapeutics. The preferred route -of injection in mammals is intravenous. It will be understood by those skilled in the art that oligonucleotides are taken up by cells and tissues in mammals and animals such as mice without special delivery methods, vehicles or solutions.

Delivery of the nucleic acid and/or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome. The recombinant retrovirus can then be used to infect and thereby deliver to the infected cells nucleic acid. The exact method of introducing the nucleic acid into mammalian cells is, of course, not limited to the use of retroviral vectors. Other techniques are widely available for this procedure including the use of adenoviral vectors, adeno-associated viral (AAV) vectors, lentiviral vectors, pseudotyped retroviral vectors. Physical transduction techniques can also be used, such as liposome delivery and receptor-mediated and other endocytosis mechanisms.

The nucleic acid or vector may be administered orally, parenterally (e.g., intravenously), by intramuscular injection, by intraperitoneal injection, transdermally, extracorporeally, intrarectally, topically or the like, although intravenous and/or intrarectal administration is typically preferred. The exact amount of the nucleic acid or vector required will vary from subject to subject, depending on the species, age, weight and general condition of the subject, the severity of the disease being treated, the particular nucleic acid or vector used, its mode of administration and the like. Thus, it is not possible to specify an exact amount for every nucleic acid or vector. However, an appropriate amount can be determined by one of ordinary skill in the art using only routine experimentation given the teachings herein (see, e.g., Remington's Pharmaceutical Sciences).

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g.,

U.S. Pat. No. 3,610,795, which is incorporated by reference herein.

Suitable carriers include, but are not limited to, pyrogen-free saline. For parenteral administration, a sterile solution or suspension is prepared in-saline that may contain additives, such as ethyl oleate or isopropyl myristate, and can be injected, for example, into subcutaneous or intramuscular tissues.

Suitable carriers for oral administration of nucleic acids include one or more substances which may also act as flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers may be water, pharmaceutically accepted oils, or a mixture of both. The liquid can also contain other suitable pharmaceutical additions such as buffers, preservatives, flavoring agents, viscosity or osmo-regulators, stabilizers or suspending agents. Examples of suitable liquid carriers include water with or without various additives, including carboxypolymethylene as a pH-regulated gel.

For example, a patient that is subject to a viral or genetic disease may be treated via in vivo or ex vivo methods. For example, for in vivo treatments, a delivery vehicle can be administered to the patient, preferably in a biologically compatible solution or a pharmaceutically acceptable carrier, by ingestion, injection, inhalation or any number of other methods. The dosages administered will vary from patient to patient. A “therapeutically effective dose” will be determined by the level of enhancement of function of the transferred genetic material balanced against any risk or deleterious side effects. Monitoring levels of gene introduction, gene expression and/or the presence or levels of the encoded anti-viral protein will assist in selecting and adjusting the dosages administered. Generally, a composition including a transfection complex will be administered in a single dose in the range of 10 ng-100 μg/kg body weight, preferably in the range of 100 ng-10 micrograms/kg body weight, such that at least one copy of the therapeutic vector is delivered to each target cell. The therapeutic vector will, of course, be associated with appropriate regulatory sequences for expression of the gene in the target cell.

Ex vivo treatment is also contemplated. Cell populations can be removed from the patient or otherwise provided, transduced with the therapeutic construct, then reintroduced into the patient. In general, ex vivo cell dosages will be determined according to the desired therapeutic effect balanced against any deleterious side-effects. Such dosages will usually be in the range of 10,000-100,000,000 cells per patient, daily weekly, or intermittently; preferably 1,000,000-10,000,000 cells per patient.

For in vitro research studies, a solution containing the vectors is added directly to a solution containing the DNA molecules of interest in accordance with methods well known to those skilled in the art and described in more detail in the examples below. In vivo research studies may be conducted by transfecting cells with plasmid DNA and incubating the vector in a solution such as growth media with the transfected cells for a sufficient amount of time for entry of the vector into the cells for triplex formation with the expressed single stranded oligonucleotide. The transfected cells may be in suspension or in a monolayer attached to a solid phase, or may be cells within a tissue wherein the vector is in the extracellular fluid. For in vitro research studies, a solution containing the vectors is added directly to a solution containing the DNA molecules of interest in accordance with methods well known to those skilled in the art.

V. Examples

The examples described below demonstrate a vector system designed to generate ssDNA in mammalian cells that was able to produce a desired 34-nucleotide TFO sequence in mouse cells, as documented by primer extension analyses performed on lysates from transfected cells. The ssDNA functions as a TFO capable of stimulating intrachromosomal recombination in a mouse cell assay that was previously shown to report triplex-induced events (Luo et al., 2000, Proc. Natl. Acad. Sci. USA, 97, 9003-9008). In particular, the combined vector set, pssXA and pssXB(AG30), specifically engineered to express the 34-nucleotide G-rich ssDNA, was found to induce recombination between the mutant TK genes in the FL-10 cells at a frequency of 196×10⁻⁶, substantially above the background level of 45×10⁻⁶. In contrast, the component vectors used individually yielded no recombination above background. When pssXA plus pssXB(rev), the latter containing the exact same insert as in pssXB(AG30) but in the reverse orientation, were used, the expected C-rich ssDNA was detected in the cells by the primer extension assay, but no induced recombination was seen. The ineffectiveness of the C-rich ssDNA is in keeping with the inability of an ODN of this sequence to form a triple helix at the polypurine target site in FL-10 cells under physiologic conditions.

Recently, Chen et al. described a vector system designed to produce ssDNA in cells, and demonstrated its use in generating either anti-sense or catalytic DNA to mediate degradation of a targeted mRNA (Chen et al., 2000, Antisense Nucleic Acid Drug Dev., 10, 415-422). In this system, co-expression from one vector of a reverse transcriptase-MboII fusion protein along with an mRNA from a second vector containing an inverted repeat sequence downstream of the Moloney murine leukemia virus (MoMuLV) core promoter is designed to yield a cDNA molecule with a stem-loop structure. MboII cleavage at the base of the stem is intended to release the ssDNA sequence of interest contained in the loop.

The ability of this vector system to produce ssDNA in mammalian cells capable of serving as a TFO for the purpose of inducing intrachromosomal events is disclosed. The disclosed ssDNA expression system produces detectable amounts of the desired TFO in cells, leading to levels of induced recombination 7-fold above that previously observed when synthetic AG30 was transfected into cells using cationic lipids (Luo et al., 2000, Proc. Natl. Acad. Sci., USA, 97, 9003-9008). The results presented herein show that the ssDNA expression system provides a useful means of generating active TFOs in vivo to mediate site-directed modification of genomic DNA in the cell in which the TFO is produced.

The present invention will be further understood by reference to the following non-limiting examples.

### EXAMPLE 1

Design of the ssDNA Vector System.

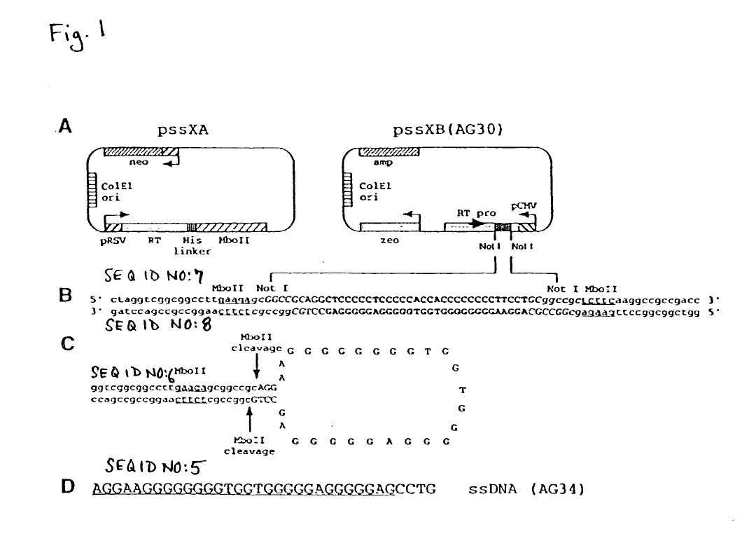

The key features of the vector system designed to produce ssDNA in cells are diagrammed in FIG. 1a (Chen et al., 2000, Antisense Nucleic Acid Drug Dev., 10, 415-422). The two-component system consists of one plasmid (pssXA) to express an RT-MboII fusion protein and a second plasmid (pssXB) to express an engineered RNA transcript from which the desired ssDNA can be generated. The reverse transcriptase can be fused to any restriction enzyme known in the art. For example, the enzyme may be selected from the non-limiting group: Eco RI, Mbo I, Hind III, Bam HI, Nde I, Bgl I, Not I, Pst I, Sac I, Ssp I, SceI and Xba I. The pssXB construct contains an expression cassette incorporating the desired insert DNA sequence (in this case a duplex incorporating the AG30 TFO sequence) flanked by inverted repeats (FIG. 1b). The resulting transcript includes an MoMuLV core promoter and tRNA primer binding site at the 3′ end (Chen et al., 2000, Antisense Nucleic Acid Drug Dev., 10, 415-422), from which RT can produce a cDNA that can form an internal stem-loop structure due to the inverted repeats (FIG. 1c). MboII cleavage of the stem is designed to release the loop as an ssDNA containing the insert sequence plus a few extraneous nucleotides. In the case of the insert incorporating the AG30 sequence, the expected 34-nucleotide ssDNA is shown in FIG. 1d. In the present work, two pssXB-derived vectors were used; one, pssXB(AG30) which has the insert sequences oriented to produce the AG34 ssDNA shown in FIG. 1d and the second having the NotI insert in the reverse orientation, thereby producing an ssDNA having a C-rich sequence with substantial but not complete complementarity to AG34 (see below).

Materials and Methods

Vectors. The construction of the pssXA and pssXB vectors (FIG. 1a) has been previously described (Chen et al., 2000, Antisense Nucleic Acid Drug Dev., 10, 415-422). The vectors contain 7869 and 5459 bp, respectively. To make vectors expressing the desired ssDNA containing the AG30 TFO sequence or the complement of it, synthetic oligonucleotides of the sequence: 5′ d(GGGCCGCAGGCTCCCCCTCCCCCACCACCCCCCCCTTCCTGC) 3′ (SEQ ID NO:1) and 5′ d(GGCCGCAGGAAGGGGGGGGTGGTGGGGGAGGGGGAGCCTGC) 3′ (SEQ ID NO:2) were annealed to produce a synthetic duplex with NotI cohesive ends and ligated into the NotI site of pssXB after removal of the stuffer fragment present in the original vector. Following transformation into E. coli, colonies were identified by direct DNA sequencing that contained plasmids with the insert sequences in both possible orientations. The orientation designed to generate a 34-nucleotide G-rich sequence incorporating the AG30 TFO is illustrated in FIG. 1 and is designated pssXB(AG30). The reverse orientation vector is designated pssXB(rev).

Oligodeoxyribonucleotides. ODNs were synthesized by the Midland Certified Reagent Co. (Midland, Tex.) and purified by either gel electrophoresis or high-pressure liquid chromatography (HPLC), followed by Centricon-3 filtration in distilled water (Amicon, Beverly, Mass.). The ODNs consisted of phosphodiester linkages and in some cases (as indicated) were synthesized to contain a 3′ propylamine group (Zendugui et al., 1992, Nucleic Acids Res., 20, 307-314).

Triplex Binding Assays. Electrophoretic mobility shift assays were performed to determine apparent dissociation constants (K_{d}s). ODNs (57 bp) containing the 30 bp polypurine TFO binding site were annealed to form a synthetic target duplex (Wang et al., 1995, Mol. Cell. Biol., 15, 1759-1768). Duplexes were radiolabeled on the 5′ end using T4 polynucleotide kinase and [γ-³²P]ATP, gel purified, and incubated for 18 hours at 37° C. with increasing concentrations of the selected TFOs in a buffer containing 10 mM Tris-HCl (pH 7.6), 1 mM spermine, and 10% glycerol. Samples were subjected to polyacrylamide gel electrophoresis in 12% native gels containing 89 mM Tris, 89 mM boric acid, pH 8.0, and 10 mM MgCl₂ for 4 hours at 60 volts, followed by autoradiography.

Cells. The construction and characterization of mouse FL-10 cells were previously described (Luo et al., Proc. Natl. Acad. Sci. USA, 97, 9003-9008). The cells were derived from LTK-cells and were determined to contain a single copy of the pTK2supF construct as a target substrate for triplex-induced intrachromosomal recombination. The FL-10 cells were grown in Dulbecco's modified Eagle's medium (DMEM) supplemented with 10% fetal bovine serum.

Vector transfection and recombination assay. FL-10 cells at a density of 1.67×10⁴ per cm² (1×10⁶ in 100 mm dishes) were transfected with 3 μg each of selected vector DNAs that were pre-mixed with 66 μl of GenePorter transfection reagent and diluted into a total of 2 ml serum-free media, as directed by the manufacturer (Gene Therapy Systems, San Diego, Calif.). After 5 hours, the medium was replaced with standard growth medium. The cells were incubated for an additional 24 hours in non-selective medium to allow recombination and expression of reconstituted TK genes to occur, after which the medium was changed to DMEM supplemented with 1×10^{31 4} M hypoxanthine, 2×10⁻⁶ M aminopterin, and 1.6×10⁻⁵ M thymidine (HAT) to select for potential recombinants expressing wild-type TK. Cells were maintained in HAT-containing medium for 10 days, at which point surviving colonies were counted.

ODN transfection. Cells at a density of 1.67×10⁴ per cm² (1×10⁶ in 100 mm dishes) were transfected with 10 μg ODN DNA per dish mixed with 66 μl of GenePorter and diluted into a total of 2 ml serum free media, as directed by the manufacturer (Gene Therapy Systems, San Diego, Calif.). As above, 5 hours after transfection the cells were placed in full growth medium supplemented with 10% FBS. Medium was changed to HAT selection 24 hours later.

Detection of ssDNA in cells. Cells were transfected with the indicated vectors using cationic lipids, as above. 24 hours later, the cells were harvested for analysis of the production of ssDNA. The cell monolayers were washed 3 times with PBS and lysed by addition of 5 ml of Trizol reagent (Life Technologies, Gaithersburg, Md.) per 60 mm dish. High molecular weight DNA in the lysate was sheared by repeated pipeting with a Pasteur pipette. The solution was transferred to a 50 ml tube, mixed with chloroform (0.2 ml per each 1 ml Trizol solution) and centrifuged for 30 min at 8000 rpm. The aqueous supernatant was mixed with 0.5 ml isopropanol per ml of Trizol initially added and centrifuged again at 5500 rpm for 30 min at 4° C. The resulting pellet was washed with 75% ethanol, air dried, and dissolved in water. RNaseA (20 μg/ml) was added and the solution was incubated at 37° C. for 2 hrs. Following phenol/chloroform extraction, the DNA was precipitated with ethanol at −70° C. and isolated by centrifugation at 14,000 rpm for 30 min at 4° C. The pellet was washed with 75% ethanol, dissolved in water, and the sample was used in a primer extension reaction. For primer extension, 25 μl of each sample was mixed with the selected primer at 80 pmole in 34 μl (radiolabeled on the 5′ end using T4 polynucleotide kinase and [γ-³²P]ATP), 2 μl each of dNTPs from 10 mM stock solutions, 2 μl vent polymerase (New England Biolabs, Beverly, Mass.), and 7 μl 10X Thermopol buffer (supplied with the vent polymerase). The reactions were heated to 95° C. for 2 min in a thermocycler and then carried out for 30 cycles: step 1, 95° C. 30 sec; step 2, 48° C. 1 min; step 3, 70° C. 2 min, followed by 10 min at 70° C. The products were visualized by electrophoresis in a 15% denaturing polyacrylamide gel followed by autoradiography. The primers were 15 mers designed to be complementary to the 3′ end of the expected products (either AG34 or rev34) and had the sequences 5′ d(CAGGCTCCCCCTCCC) 3′ (SEQ ID NO:3) and 5′ d(CAGGAAGGGGGGGGT) 3′ (SEQ ID NO:4), respectively.

Quantification of ssDNA in transfected cells. 24 h following transfection, the cells were washed 3 times with PBS and lysed for preparation of low molecular weight DNA (as above). At the same time, parallel samples were spiked with synthetic AG34 ODN (the predicted product of pssXA and pssXB(AG30)) at concentrations designed to mimic from 10⁴ to 10⁷ molecules per cell, assuming approximately 4×10⁶ cells are present at the time of lysis. The rest of the analysis via primer extension was as above.

### EXAMPLE 2

Comparative Binding of Synthetic TFOs and Potential ssDNA Products.

In the expected stem loop structure within the cDNA product of the vector system, the cleavage by MboII occurs within the stem (FIG. 1c). Maintenance of the stem to preserve MboII cleavage site requires that the insert sequences incorporate complementary nucleotides at the 5′ and 3′ ends. In the case of the AG30 sequence, this meant that, in the ssDNA ultimately produced, 4 extra nucleotides would have to be included at either the 5′ or 3′ end. Although previous work had shown that the AG30 TFO binds with high affinity to the polypurine target site in the FL-10 cell recombination substrate, we were concerned that extra nucleotides at either the 5′ or 3′ end of the TFO might substantially diminish binding. To determine the effect of either a 5′ or 3′ tail on AG30 third-strand binding, gel mobility shift assays were carried out using a synthetic 57 bp duplex as a binding target. For comparison, we also tested binding by the AG30 TFO with a 3′ propylamine (as used in previous targeting experiments in mouse cells and mice (Luo et al., 2000, Proc. Natl. Acad. Sci. USA, 97, 9003-9008; Vasquez et al., 2000, Science, 290, 530-533) and by the AG30 TFO with a 3′ OH, since the ssDNA expected to be produced intracellularly by the vector system would have a 3′ OH. As shown, both AG30 with a 5′ tail and AG30 with a 3′ tail show reduced binding relative to AG30, with equilibrium dissociation constants (K_{d}s) in the range of 5×10⁻⁷ M versus 5×10⁻⁸ M for AG30. Hence, the 4 nucleotide tails reduce but do not eliminate third-strand binding affinity. In the pssXB(AG30) construct used in our experiments, the predicted ssDNA product will have a 3′ tail relative to the AG30 sequence (FIG. 1d).

### EXAMPLE 3

Assay for Induced Intrachromosomal Recombination in Mouse Cells.

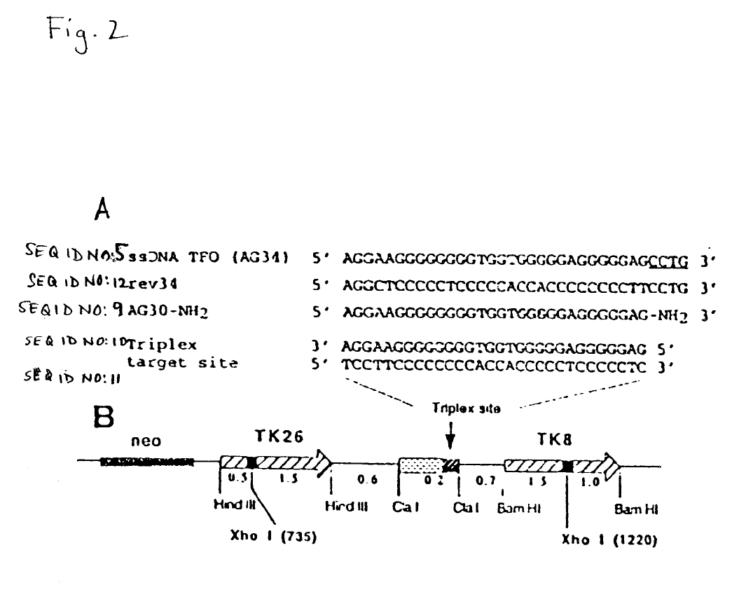

An assay for triplex-induced intrachromosomal recombination was used to test the ability of the vector system to produce ssDNA capable of acting as a TFO to bind to a chromosomal target site. A subclone of mouse LTK-cells (FL-10) carries a pair of mutant TK genes in a single locus as direct repeats (FIG. 2). In this construct, the region between the TK genes was engineered to contain the 30 bp G-rich polypurine sequence amenable to high-affinity third-strand binding in the anti-parallel triplex motif (Beal et al., 1991, Science, 251, 1360-1363) by the AG30 TFO (Wang et al., 1995, Mol. Cell. Biol., 15, 1759-1768). The TK genes contain inactivating XhoI linker insertion mutations at different sites (positions 735 in TK 26 and 1220 in TK8). In the assay, recombination between the two TK genes has the potential to produce a functional gene. Since the parental LTK-cells lack the cellular TK, cells in which the mutant TK genes have recombined to generate a wild-type TK can be selected by growth in the presence of HAT medium. Induction of recombination by transfection with selected vectors or ODNs is quantified by enumerating the HAT-resistant colonies as a proportion of the total number of cells treated. In previous work, the recombination substrate in the FL-10 cells is biased toward reporting gene conversion events rather than crossover recombination (Luo et al., 2000, Proc. Natl. Acad. Sci. USA, 97, 9003-9008), and so the assay may actually underestimate the frequency of triplex-induced events.

### EXAMPLE 4

Induced Recombination by ssDNA Vectors.

The FL-10 cells were transfected with a series of vectors, either individually or in pairs, and induction of HAT-resistant colonies was assayed in at least three separate experiments. The background frequency of recombination in this set of experiments was in the range of 45×10⁻⁶, and no induction above this level was seen when either pssXA, pssXB, or pssXA plus pssXB were transfected into the cells. However, when pssXA plus pssXB(AG30) were co-transfected, recombinants were produced at a frequency of 196×10⁻⁶. No effect was seen when pssXA was combined with pssXB(rev), containing the AG30 sequence insert in reverse orientation. This vector set is expected to express the C-rich rev34 ssDNA (FIG. 2) which does not form triplex at the polypurine target site under physiologic pH conditions due to the need for cytosine protonation.

Subtracting the background of spontaneous recombination in the assay, transfection of the FL-10 cells with pssXA and pssXB(AG30) yielded, on average, an induced frequency of recombinants of 151 in 10⁶. For comparison, in previous work, when AG30-NH₂ (end-protected with a propylamine substitution for the 3′ OH) was transfected into the same cells by cationic lipids, the induction above background was 21 in 10⁶ (Luo et al., 2000, Proc. Natl. Acad. Sci. USA, 97, 9003-9008). For further comparison, a synthetic version of AG34 (designed to match the predicted ssDNA molecule and therefore made without propylamine protection) did not induce recombination when transfected into the FL-10 cells using cationic lipids. Taken together, these results indicate that the use of the ssDNA system for intracellular generation of AG34 inside cells is substantially more effective in achieving chromosome targeting than is lipid-mediated transfection with the synthetic TFOs, either AG30-NH₂ or AG34. In addition, the results demonstrate that, as expected, a synthetic version of AG34 is less active than AG30 when transfected into the FL-10 cells. This is consistent with the reduced binding affinity of AG34 versus AG30 for the 30 bp polypurine target site in these cells and also likely reflects the lack of end-protection on AG34 to block nuclease degradation.

### EXAMPLE 5

Detection of ssDNA Products in Cells.

To confirm that the expected ssDNA molecules were generated by the transfected vectors, low molecular weight DNA was isolated from the cells 24 hours following vector transfection and carried out a primer extension assay using primers radiolabeled at the 5′ end. Lysates obtained from untransfected cells and from cells transfected with pssXA alone, pssXB alone, pssXA plus pssXB, or with pssXA plus pssXB(AG30) were assayed using a 15-nucleotide primer designed to be complementary to the AG34 sequence (FIGS. 1d). The lysate from cells transfected with pssXA and pssXB(rev) was assayed with a 15-nucleotide primer designed to detect the rev34 sequence (FIG. 2). As shown, 34-nucleotide ssDNA species were visualized only in lysates from cells transfected by pssXA plus pssXB(AG30) or pssXA plus pssXB(rev), using the AG34- and rev34-specific primers, respectively. Only transfection with pssXA plus pssXB(AG30) yielded induced recombinants.

### EXAMPLE 6

Quantification of ssDNA Production in Cells.

To determine the approximate yield of ssDNA molecules produced per cell by the vector set, pssXA and pssXB(AG30), mouse FL-10 cells were transfected either with pssXA plus pssXB(AG30) (via co-mixture with cationic lipids) or, for comparison, with a synthetic ODN, AG34 (also by lipofection). The AG34 oligomer was 3′ end-protected with propylamine but was otherwise designed to exactly match the predicted product of the pssXA plus pssXB(AG30) vectors. As above, low molecular weight DNA was isolated from the cells 24 hours following transfection, and a primer extension assay was used to visualize and quantify the ssDNA (FIG. 6). At the time of lysis, parallel samples were spiked with known quantities of AG34 at concentrations calculated to mimic from 10⁴ to 10⁷ molecules per cell. The data was quantified by phosphorimager, and the standard curve was determined by linear regression. The quantity per cell of ssDNA molecules in the experimental samples was estimated by interpolation from the standard curve, yielding values of 6.2×10⁵ molecules per cell for the pssXA plus pssXB(AG30) sample and 1.9×10⁵ for the AG34 sample.

Previously, it was not only established that transfected TFOs could induce recombination within the dual TK substrate in the FL-10 cells (Luo et al., 2000, Proc. Natl. Acad. Sci. USA, 97, 9003-9008), but also that the assay could report induced recombination over a range of frequencies, from 10⁻⁶ to 10⁻². Importantly, the prior work revealed that the level of TFO-induced recombination was very much dependent on the efficiency of intracellular delivery of the TFOs. When the AG30 TFO was transfected using cationic lipids, a frequency of induced recombination of 21×10⁻⁶ above background was seen (Luo et al., 2000, Proc. Natl. Acad. Sci. USA, 97, 9003-9008). When the TFOs were introduced by microinjection, recombination frequencies in the range of 1% were detected.

In the work reported here, pssXA and pssXB(AG30) were co-transfected by mixture with cationic lipids, and the average induction above background was 151×10⁻⁶. This result can be correlated with quantification of intracellular ssDNA production based on a primer extension assay, which yielded an estimate of 6.2×10⁵ molecules per cell generated by the pssXA plus pssXB(AG30) vector set. In contrast, transfection of the synthetic ODN, AG34, by lipofection yielded approximately 1.9×10⁵ molecules per cell. The relative level of ssDNA production by the vector set is substantial, considering that, in these experiments, the co-transfection of the two vectors was not optimized; and so it is likely that a sizable number of the cells were not transfected with both vectors.

Modifications to the vector system, such as consolidation of the key components (for example, the nucleic acid encoding the reverse transcriptase-restriction enzyme fusion and the nucleic acid encoding the single stranded triplex forming oligonucleotide) into a single plasmid and incorporation into a viral vector, improves transfection efficiency and leads to increased activity. In relative terms, the yield of recombinants generated by microinjection of the synthetic AG30 TFO into the FL-10 cells in previous work (Luo et al., 2000, Proc. Natl. Acad. Sci. USA, 97, 9003-9008) was 66-fold higher than that induced by pssXA plus pssXB(AG30) in the work here. On the other hand, the ssDNA vector system was found to produce recombinants above background at a frequency 7-fold higher than that stimulated by AG30 transfection using cationic lipids [data also from the previous study (Luo et al., 2000, Proc. Natl. Acad. Sci. USA, 97, 9003-9008)]. The estimated numbers of ODNs or ssDNA molecules generated per cell by these methods are 7×10⁴, 6×10⁵, and 1.9×10⁵, respectively.

A synthetic 34-mer matching the expected ssDNA product has a binding affinity about 10-fold lower than that of AG30 itself. That the ssDNA system was still effective in inducing recombination in spite of this decreased affinity further serves to demonstrate the power of the system, and it suggests that substantial increases in TFO activity is possible when the extra nucleotides are eliminated. In addition, this difference in affinity partially explains why microinjection of the precise AG30 molecule is more effective than the ssDNA vector system (Luo et al., 2000, Proc. Natl. Acad. Sci. USA, 97, 9003-9008). The data described herein measures induced recombination and documents the production of predicted ssDNA species. Based upon Chen et al. (Chen et al., 2000, Antisense Nucleic Acid Drug Dev., 10, 415-422), who first reported this approach for anti-sense DNA generation, the ssDNA species is derived from the activity of reverse transcriptase (see Chen et al., wherein they provide evidence of RT activity in the vector-transfected cells).

While this is the first use of a vector system to produce ssDNA in cells for chromosomal triplex formation, some previous studies have explored the use of intracellularly generated RNA transcripts for this purpose (Noonberg et al., 1994, Nucleic Acids Res., 22, 2830-2836; Shevelev et al., 1997, Cancer Gene Therapy, 4, 105-112). However, the RNA-based approach has several disadvantages. RNA cannot recombine with DNA. Naturally occurring RNA is excluded from the anti-parallel purine motif for triplex formation that is otherwise favored at G:C bp-rich target sites, such as the one used in the experiments here (Beal et al., 1991, Science, 251, 1360-1363; Roberts et al., 1992, Science, 258, 1463-1466; Semerad et al., 1994, Nucleic Acids Res., 22, 5321-5325). On the other hand, triplex formation in the parallel pyrimidine motif by naturally occurring RNA or DNA requires acidic pH because of the need for cytosine protonation (Asensio et al., 1998, J. Mol. Biol., 275, 811-822; Singleton et al., 1992, Biochemistry, 31, 10995-1003). Strategies to overcome this require chemical modification of the C residues, which cannot be accomplished in the case of biologically generated molecules. In addition, no mechanism has yet been developed to post-transcriptionally modify the RNAs (in a manner analogous to the use of the MboII activity here), and so the transcripts typically carry a large number of extra nucleotides that reduce the third-strand binding affinity. The use of engineered RNA transcripts generated inside cells, on the other hand, has shown substantial promise for anti-sense applications (Gorman et al., 1998, Proc. Natl. Acad. Sci. USA, 95, 4929-4934).

In conclusion, the results presented here demonstrate that ssDNA can be effectively generated inside mammalian cells for the purpose of creating TFOs to target chromosomal sites. Other applications of triplex technology, such as targeted gene knockout or transcription inhibition, can be accommodated using the approaches described herein. The ability to generate anti-gene TFOs with high efficiency in mammalian cells offers an important new research tool and provides the basis of a novel form of therapy.

Those skilled in the art will recognize, or be able to ascertain using no more than routine experimentation, many equivalents to the specific embodiments of the invention described herein. Such equivalents are intended to be encompassed by the following claims.

I claim:

1. A method for inducing a specific change in a target chromosomal nucleic acid molecule comprising the steps of:
(a) introducing into a cell a nucleotide molecule encoding a reverse transcriptase; and
(b) introducing into the cell a nucleotide molecule encoding a RNA to be reverse transcribed into single stranded DNA by the reverse transcriptase in the cell;
wherein the single stranded DNA binds to the target chromosomal nucleic acid molecule in the cell to form a triplex to induce a site specific change and/or mediate recombination with the target chromosomal nucleic acid molecule.

2. The method of claim 1, wherein the single stranded DNA mediates recombination with the target.

3. The method of claim 1, wherein the triplex formation induces recombination.

4. The method of claim 1, wherein the triplex induces mutation without recombination.

5. The method of claim 3, wherein the recombination is intra-chromosomal.

6. The method of claim 3, wherein the recombination is inter-chromosomal.

7. The method of claim 1 wherein the RNA is reverse transcribed into a DNA that forms a double stranded stem-single stranded loop structure, wherein the double stranded stem is cleaved away from the single stranded loop structure by a restriction enzyme introduced with the reverse transcriptase.

8. The method of claim 7, wherein the restriction enzyme is selected from the group consisting of Mbo II, Sce I, Eco RI, Mbo I, Hind III, Bam HI, Nde I, Bgl I, Not I, Pst I, Sac I, Ssp I, SceI and Xba I.

9. The method of claim 7, wherein the restriction enzyme is a rare cutting restriction enzyme.

10. The method of claim 9, wherein the rare cutting enzyme is Sce I.

11. The method of claim 7, wherein the triplex induces recombination.

12. The method of claim 11, wherein the recombination is intra-chromosomal.

13. The method of claim 11, wherein the recombination is inter-chromosomal.

14. The method of claim 7 wherein the reverse transcriptase and restriction enzyme are in a fusion protein.

15. The method of claim 11 wherein the single stranded DNA stimulates recombination of an exogenously supplied DNA fragment with the target chromosomal sequence.

16. The method of claim 11 wherein the single stranded DNA stimulates recombination of a tethered DNA fragment with the target chromosomal sequence.

17. The method of claim 1 wherein the target chromosomal gene is an oncogene.

18. The method of claim 1 wherein the target chromosomal gene is a defective gene selected from the group of genes consisting of a defective β-hemoglobin gene, hemophilia, cystic fibrosis gene, xeroderma pigmentosum gene, nucleotide excision repair pathway gene and hemophilia gene.

19. The method of claim 1 wherein the target chromosomal sequence is all or a portion of a viral genome.

20. The method of claim 1 wherein the single stranded DNA is composed of homopurine or homopyrimidine nucleotides.

21. The method of claim 1 wherein the single stranded DNA is composed of polypurine or polyrimidine nucleotides.

22. The method according to claim 1, wherein the reverse transcriptase and restriction enzyme and the RNA are expressed from two or more vectors.

23. The method according to claim 1, wherein the reverse transcriptase, restriction enzyme, and RNA are expressed from the same vector.

24. The method of claim 1, further comprising:
(c) introducing into the cell a nucleic acid encoding a recombinagenic DNA donor fragment.

25. The method of claim 1, further comprising:
(c) introducing into the cell a synthetically derived recombinagenic DNA donor fragment.

26. An expression system that generates single stranded DNA in a cell, wherein the single stranded DNA binds to a target chromosomal sequence in the cell, comprising
(a) a nucleotide molecule that encodes a reverse transcriptase; and
(b) a nucleotide molecule that encodes a RNA that is reverse transcribed into the single stranded DNA by the reverse transcriptase.

27. The expression system of claim 26, wherein the single stranded DNA binds to the target chromosomal sequence to form a triplex.

28. The expression system of claim 26, wherein the single stranded DNA is a recombinagenic donor DNA molecule.

29. The expression system of claim 26 comprising a triplex forming single stranded DNA and a recombinagenic single stranded DNA molecule.

30. The expression system of claim 26, further encoding a restriction enzyme, the system comprising a nucleotide molecule encoding a RNA to be reverse transcribed into a DNA that forms a double stranded stem-single stranded loop structure, wherein the double stranded stem is cleaved away from the single stranded loop structure by the restriction.

31. The expression system of claim 30 wherein the reverse transcriptase and restriction enzyme are expressed as a fusion protein.

32. The expression system of claim 26, wherein the RNA, the reverse transcriptase, and the restriction enzyme are expressed from the same nucleotide molecule.

33. The expression system of claim 26, wherein the RNA, the reverse transcriptase, and the restriction enzyme are expressed from two ore more separate nucleotide molecules.

34. The expression system of claim 26, wherein the DNA encoding the RNA and the DNA encoding the reverse transcriptase-restriction enzyme fusion are integratable into the chromosome.

35. The expression system of claim 30, wherein the restriction enzyme is selected from the group consisting of Mbo II, Sce I, Eco RI, Mbo I, Hind III, Bam HI, Nde I, Bgl I, Not I, Pst I, Sac I, Ssp I, SceI and Xba I.
